(19) United States
(12) Patent Application Publication
(10) Pub. No.: US 2009/0036813 A1
(43) Pub. Date: Feb. 5, 2009

(54) APPARATUS AND METHOD FOR ASSISTING STERNAL REHABILITATION

(76) Inventor: Wendy Whitney, Salt Lake City, UT (US)

Correspondence Address: Kunzler & McKenzie, 8 EAST BROADWAY, SUITE 600, SALT LAKE CITY, UT, 84111, US

(21) Appl. No.: 12/180,686

(22) Filed: Jul. 28, 2008

Related U.S. Application Data
(60) Provisional application No. 60/952,824, filed on Jul. 30, 2007.

(51) Int. Cl. A61F 5/00; A61F 5/37

(52) U.S. Cl. 602/20; 128/878

(57) ABSTRACT

The apparatus to assist sternal rehabilitation includes a torso belt circumscribing an abdomen of a patient, a first humeral restraint coupled to the torso belt, and a second humeral restraint coupled to the torso belt. Each humeral restraint circumscribes an upper arm of the patient between a glenohumeral joint and an antecubital space of the upper arm. The humeral restraints substantially confine the upper arms to a position lateral, slightly anterior, and to the side of a torso of the patient. The torso belt may include a substantially inelastic flexible material. The torso belt may also include a comfort material coupled to the substantially inelastic flexible material. The comfort material may be interposed between the substantially inelastic flexible material and the torso. The torso belt may circumscribe the abdomen below a pectoral line and above a pelvic line of the patient.

### CROSS-REFERENCES TO RELATED APPLICATIONS

This application claims the benefit of U.S. Provisional Patent Application No. 60/952,824 entitled “APPARATUS FOR ASSISTING STERNAL REHABILITATION” and filed on Jul. 30, 2007 for Wendy Whitney, which is incorporated herein by reference.

### FIELD OF THE INVENTION

This invention relates to post surgery rehabilitation devices and more particularly relates to a sternal brace for assisting successful rehabilitation of the sternum following a sternotomy.

### BACKGROUND

### Description of the Related Art

Sternotomies are a necessary surgical procedure to gain access to the chest cavity during surgeries that involve the heart or lungs. The sternum is a broad bone, commonly referred to as the breast bone, which protects the heart and supports the ribs. After a sternum is cut during surgery, it is typically wired together with sternal wires so that the edges meet to provide bone healing. If the wires break, a space forms between the bone edges. Fluids may fill this space and cause infection. Recovery after a wire breaks may take up to one year.

If intravenous antibiotics do not cure a patient's sternal infection, the patient may require a sternectomy to remove the entire sternum. A sternectomy is a debilitating surgery because a muscle flap, usually the pectoris muscle, must be cut and sewn upward where the sternum previously protected the patient's heart. The patient may have to wear a plastic breast cover to protect the patient's heart and chest. The patient must take precautions to not be hit or bumped in the chest because he has a flexible muscle protecting his heart instead of an inflexible bone.

Actions that may cause the sternal wires to break apart are those that require a force to be applied by the muscles of the upper arms. One common action a sternotomy patient should avoid is using his arms to push or pull himself out of a chair. Another damaging action occurs when a patient tries to lift something with his elbows abducted from his torso. Some patients are demented or delusional after surgery and push down with their elbows on the bed. This action causes pressure on the sternal wires and may cause them to break.

To avoid these actions, a patient may need to have his entire arms restrained while the sternum heals. This option is undesirable because it renders the patient unable to use his arms at all for several weeks. A patient who has his arms entirely restrained requires constant care to accomplish basic daily tasks such as eating and bathing. If he is demented or delusional, the patient should be restrained in such a way as to not allow his elbows to push down on the bed. Even if a patient is not demented or delusional, it is often difficult to be mindful of what he is doing with his arms at all times. Sternotomy is a painful surgery, and most patients are given narcotics to eliminate pain. Narcotics often prevent patients from following directions at all times.

Sternotomy patients are typically instructed to consciously avoid the damaging actions for several weeks. The problem with this method is that patients are accustomed to using the full range of their upper arms and often move their arms without consciously realizing what they are doing. If the muscles of the upper arms are moved from the side of the torso and used, even accidentally, the patient risks breaking the sternal wires and the complications discussed above.

### SUMMARY

From the foregoing discussion, it should be apparent that a need exists for an apparatus and method that assists sternal rehabilitation. Beneficially, such an apparatus and method would provide patients substantial use of their arm for activities that are not damaging to the healing process. Potentially damaging arm movements would be restricted.

The present invention has been developed in response to the present state of the art, and in particular, in response to the problems and needs in the art that have not yet been fully solved by currently available restraint methods. Accordingly, the present invention has been developed to provide an apparatus and method for assisting sternal rehabilitation that overcome many or all of the above-discussed shortcomings in the art.

The apparatus to assist sternal rehabilitation includes a torso belt circumscribing an abdomen of a patient, a first humeral restraint coupled to the torso belt, and a second humeral restraint coupled to the torso belt. The first humeral restraint circumscribes a first upper arm of the patient between a glenohumeral joint and an antecubital space of the first upper arm. The first humeral restraint substantially confines the first upper arm to a position lateral, slightly anterior, and to the side of a torso of the patient.

The second humeral restraint circumscribes a second upper arm of the patient between a glenohumeral joint and an antecubital space of the second upper arm. The second humeral restraint substantially confines the second upper arm to a position lateral, slightly anterior, and to the side of the torso of the patient.

The torso belt may include a substantially inelastic flexible material. The torso belt may also include a comfort material coupled to the substantially inelastic flexible material. The comfort material may be interposed between the substantially inelastic flexible material and the torso.

The torso belt may circumscribe the abdomen below a pectoral line and above a pelvic line of the patient. Each humeral restraint may position a midpoint of each corresponding upper arm between a vertical mid-axillary line and a vertical line through a greater trochanter of a femur.

The torso belt may comprise at least one belt fastener. The belt fastener secures the torso belt to the abdomen of the patient. The length of the torso belt may be adjustable.

Each of the humeral restraints may include a substantially inelastic flexible material. Each of the humeral restraints may also include a comfort material coupled to the substantially inelastic flexible material. The comfort material may be interposed between the substantially inelastic flexible material and each upper arm.

Each of the humeral restraints may be coupled to the torso belt with a restraint fastener. The restraint fastener may anchor each of the humeral restraints to the torso belt, thereby substantially securing the upper arm to the side of the torso. The length of each of the humeral restraints may be adjustable. Each of the humeral restraints may include a cuff circumscribing the corresponding upper arm secured with Velcro® or other hook and loop closure system.

In one embodiment, the apparatus to assist sternal rehabilitation includes a torso belt circumscribing an abdomen of a patient. The torso belt includes a substantially inelastic flexible material, and the torso belt circumscribes the abdomen between a pectoral line and a pelvic line of the patient. The apparatus also includes a first humeral restraint and a second humeral restraint coupled to the torso belt. Each humeral restraint circumscribes an upper arm of the patient between a glenohumeral joint and an antecubital space of the upper arm. Each humeral restraint substantially confines the upper arm to a position lateral, slightly anterior, and to the side of a torso of the patient. Each humeral restraint includes a substantially inelastic flexible material.

The method for assisting sternal rehabilitation includes circumscribing a torso belt about an abdomen of a patient, circumscribing a first humeral restraint about a first upper arm, restraining the first humeral restraint to the torso belt, circumscribing a second humeral restraint about a second upper arm, and restraining the second humeral restraint to the torso belt. The first humeral restraint circumscribes a first upper arm of the patient between a glenohumeral joint and an antecubital space of the first upper arm. Restraining the first humeral restraint to the torso belt substantially confines the first upper arm to a position lateral, slightly anterior, and to the side of a torso.

The second humeral restraint circumscribes a second upper arm of the patient between a glenohumeral joint and an antecubital space of the second upper arm. Restraining the second humeral restraint to the torso belt substantially confines the second upper arm to a position lateral, slightly anterior, and to the side of a torso.

In an embodiment of the method, the torso belt may include a substantially inelastic flexible material. A comfort material may be interposed between the substantially inelastic flexible material and the torso. The torso belt may circumscribe the abdomen below a pectoral line and above a pelvic line of the patient. A midpoint of each upper arm may be restrained between a vertical mid-axillary line and a vertical line through a greater trochanter of a femur. Each humeral restraint may include a substantially inelastic flexible material. A comfort material may be interposed between the substantially inelastic flexible material and each upper arm.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore considered limiting of its scope, the invention will be described and explained with additional specificity and detail through the use of the accompanying drawings, in which:

FIG. 1 is a front view of one embodiment of a sternal brace in accordance with the present invention;

FIG. 2 is a side view of one embodiment of the sternal brace in accordance with the present invention;

FIG. 3 is a rear view of one embodiment of the sternal brace in accordance with the present invention;

FIG. 4 is another view of one embodiment of the sternal brace in accordance with the present invention; and

FIG. 5 is a more detailed depiction of an adjustable cuff of an adjustable humeral restraint in accordance with the present invention.

FIG. 6 is a schematic flow chart diagram illustrating an embodiment of a method for assisting sternal rehabilitation in accordance with the present invention.

### DETAILED DESCRIPTION

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

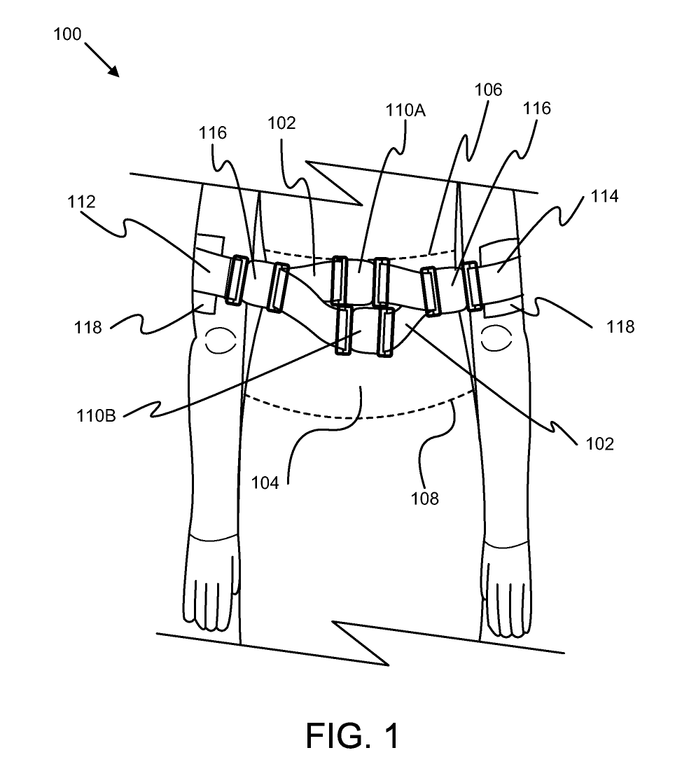

FIG. 1 is a front view of one embodiment of a sternal brace 100 in accordance with the present invention. The sternal brace 100 includes a torso belt 102 circumscribing an abdomen 104. In one embodiment, the torso belt 102 circumscribes the abdomen 104 below a pectoral line 106 and above a pelvic line 108. The pectoral line 106 is a line along the bottom of the pectoralis major muscles in the abdomen. The pelvic line 108 is a line along the top of the pelvis. In a preferred embodiment the torso belt 102 may be fitted high enough on an abdomen 104 such that the torso belt 102 is connected to humeral restraints 112, 114 that are properly positioned around a pair of upper arms, and low enough to not restrict the breathing of a patient.

In one embodiment the torso belt 102 may be adjustable. The torso belt 102 may comprise at least one type of substantially inelastic flexible material such as canvas, plastic, nylon, polypropylene, a polymer, a monomer, and/or other material configurable to be substantially inelastic and flexible. For example, the material of the torso belt 102 should be flexible to wrap around the abdomen 104 and substantially inelastic to resist stretching when pulled and twisted. In alternate embodiments the torso belt 102 may comprise other types and configurations of straps including shoulder straps and/or straps that pass between the legs to hold the torso belt 102 in place. The torso belt 102 may further comprise a non-slip material that may further assist holding the torso belt 102 in place.

The torso belt 102 may comprise at least one belt fastener 110 securing the torso belt to the abdomen 104. The depicted embodiment shows two belt fasteners 110A, 110B. Alternate embodiments of the torso belt 102 may comprise one belt fastener 110. The belt fastener 110 may comprise a clip and/or a snap type mechanism suitable for easily securing the torso belt 102 about the abdomen 104. In the depicted embodiment, the belt fastener 110 further comprises a common friction loop belt adjuster where a user may securely change the length of the torso belt 102 for a given application, though other belt adjustable devices such as wraps with a hook and loop closure system such as Velcro®, zippers, buttons, ties, snaps, clips, clasps, and/or other types of fastening devices may be used.

Any type of torso belt 102 may be used that is substantially inelastic. The torso belt 102 may further comprise a comfort material coupled to the substantially inelastic flexible material with the comfort material interposed between the substantially inelastic flexible material and the torso. The comfort material may comprise at least one type of material such as cotton, felt, velvet, linen, silk, nylon, acetate rayon, and/or other material suitable for prolonged contact with a human body.

The front view in FIG. 1 further shows a first humeral restraint 112 and a second humeral restraint 114 coupled to the torso belt 102, each humeral restraint 112, 114 circumscribing an upper arm below a glenohumeral joint and above an antecubital space. The glenohumeral joint comprises the joint formed by the connection of the upper arm to the shoulder, and the antecubital space is the region at the front of the arm formed by the connection of the upper arm to the lower arm on an opposite side of the arm from the elbow. In one embodiment, each humeral restraint 112, 114 comprises adjustable humeral restraints 112, 114. Each humeral restraint 112, 114 further comprises at least one type of substantially inelastic flexible material. For example, each humeral restraint 112, 114 may be manufactured of the same material as the torso belt 102.

The front view in FIG. 1 further illustrates each of the humeral restraints 112, 114 coupled to the torso belt 102 further comprising a restraint fastener 116. For example, the humeral restraint 114 maybe coupled to the torso belt 102 at a location behind the upper arm and then further fastened to the torso belt 102 by the restraint fastener 116 at a location in front of the upper arm. In one embodiment, the restraint fasteners 116 may comprise the common friction loop type of fastener used for the torso belt 102. In other embodiments, the humeral restraints 112, 114 may comprise wraps of Velcro®, zippers, buttons, ties, snaps, clips, clasps and/or other types of fastening devices. In one embodiment, coupling to the rear of the upper arm and fastening to the front of the upper arm by the humeral restraints 112, 114 facilitates the anchoring of the humeral restraints 112, 114 to the torso belt 102 and thereby substantially secures the upper arm to the side of the torso.

In an alternate embodiment the humeral restraints 112, 114 may be substantially anchored to the torso belt 102 and comprise no restraint fasteners 116 as the humeral restraints 112, 114 may slide up the arm from the hand. In one embodiment the humeral restraints 112, 114 may be detachable from the torso belt 102 by means of ties, clips, zippers, clasps, and/or other fastening devices suitable for preventing movements of the upper arm away from the torso during use. In an alternate embodiment the humeral restraints 112, 114 may be permanently affixed to the torso belt 102. For example, the humeral restraints 112, 114 may be formed together with the torso belt 102 during manufacture, stitched, sewn, adhered, and/or otherwise permanently coupled to the torso belt 102.

For successful and efficient sternal rehabilitation, movements of the upper arms must be necessarily very limited. Certain movements of the upper arms and exertions of the muscles in the upper arms may split the sternum. For example, simple tasks such as pushing oneself up from a chair where the upper arms rotate to an anterior position of the torso, or pulling oneself up to a table may be especially harmful. The present invention may permit a range of movement of the lower arms such as for grooming, eating, and other tasks while preventing movements that would increase the risk of separating the sternum while the sternum is healing. By keeping the upper arms close to the torso and slightly forward of the midaxillary line, the sternal brace 100 helps to prevent risky movement that has a tendency to separate a sternum that is healing after a sternotomy.

A key objective of the present invention is to prevent movements of the upper arms that may create stress on the sternum. In one example of using the sternal brace 100, the torso belt 102 is fastened loosely about the abdomen and then tightened to securely circumscribe the abdomen, and each upper arm is wrapped securely above the antecubital space in a cuff of each of the humeral restraints 112, 114 secured with Velcro® or a similar closure system. In the continuing example, with the upper arms placed in the Velcro® cuffs of the humeral restraints 112, 114, and the humeral restraints 112, 114 in a position to the side and slightly anterior of the torso, the humeral restraints 112, 114 are fastened loosely and then tightened to anchor the upper arms securely to the torso belt 102 keeping the upper arms in secure and constant contact with the side of the torso.

Because the sternum is a vital structural and functional element of the human body which directly or indirectly anchors multiple bones and muscles of the upper body, it is vital that movements of the upper body be necessarily limited during the healing period of the sternum. For a patient recovering from a sternotomy it may be very challenging to cease simple and natural movements of the upper arms that may permanently damage the sternum. The use of the sternal brace 100 during recovery provides a means to hold the upper arms in an advantageous healing position, restrict movements of the upper arms that may cause stress and/or damage to the sternum, and allow for movements of the lower arms to accomplish everyday tasks such as eating. The sternal brace 100 may help the patient keep his arms close to the torso, slightly anterior, to protect the sternal wires. If the patient pulls with his hands, the force may be transferred to the bicep instead of the pectoral muscle of the chest. This may help keep the sternal wires intact.

In one embodiment, the humeral restraints 112, 114 comprise a comfort material 118 coupled to the substantially inelastic flexible material. The comfort material 118 may be interposed between the substantially inelastic flexible material and the upper arm.

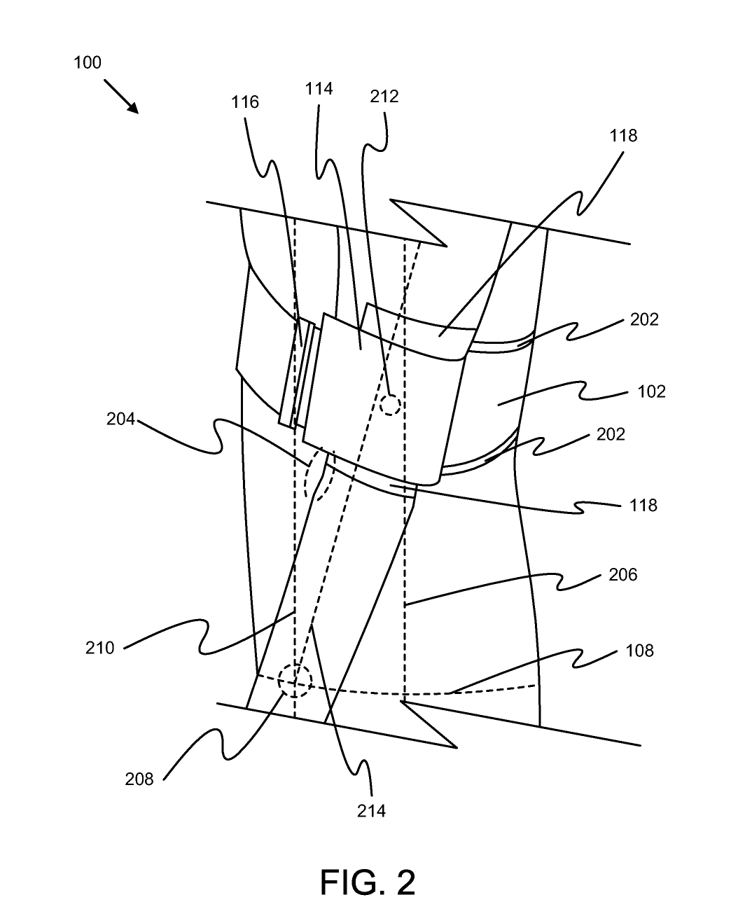

FIG. 2 is a side view of one embodiment of the sternal brace 100 in accordance with the present invention. The side view shows the torso belt 102 with the corresponding comfort material 202 interposed between the torso belt 102 and torso. In one embodiment, the comfort material 202 may comprise the same comfort material 118 for the humeral restraints 112, 114.

FIG. 2 further depicts the humeral restraint 114 and the corresponding comfort material 118 and the restraint fastener 116. The antecubital space 204 is the area on the arm where the elbow bends. As the humeral restraint 114 is secured to the upper arm above the antecubital space 204, movement of the lower arm may be permitted while the upper arm is restrained.

Preferably, the humeral restraint 114 is positioned such that the midpoint 212 of the patient's upper arm is positioned on or forward of the mid-axillary line 206 that runs down the center of the torso. In addition, the humeral restraint 114 is positioned such that the midpoint 212 of the patient's upper arm is at or behind a vertical line 210 passing through the greater trochanter 208. The greater trochanter 208 is located at the head of the femur and is shown in FIG. 2 as a point at the end of a horizontal line at the patient's pelvic line 108. In the embodiment shown in FIG. 2, the humeral restraint 114 positions the arm so that a line 214 drawn through the humerus bone in the upper arm runs approximately through the greater trochanter 208. Typically, the arm is positioned to the side of the torso by the humeral restraint 114 between the mid-axillary line 206 and the vertical line 210 through the greater trochanter 208.

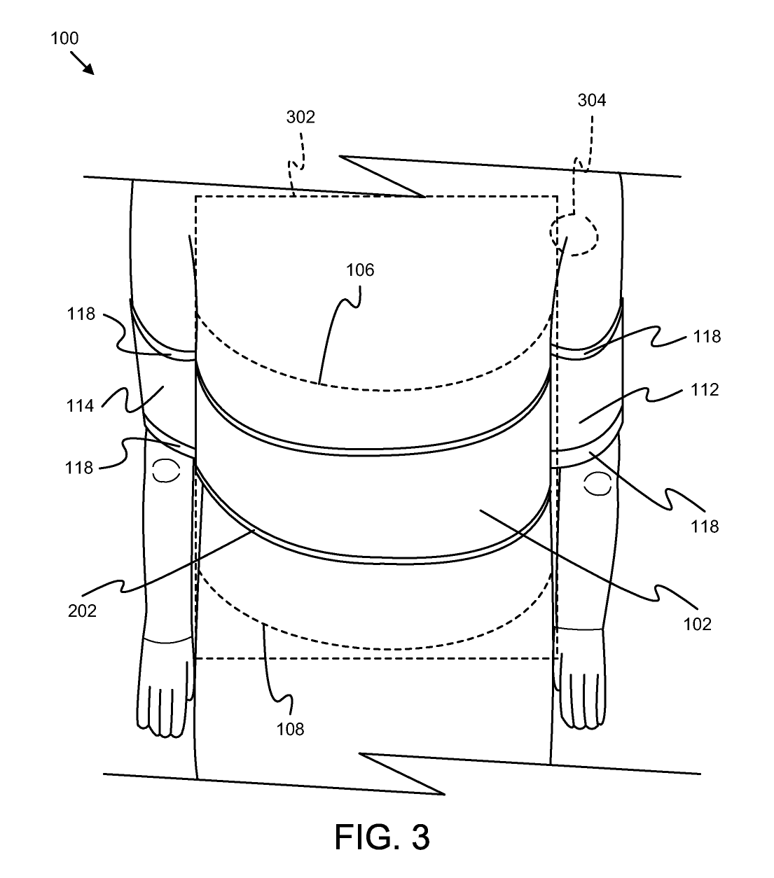

FIG. 3 is a rear view of one embodiment of the sternal brace 100 in accordance with the present invention. FIG. 3 shows the humeral restraints 112, 114, and the corresponding comfort material 118. FIG. 3 further illustrates the rear view of the torso belt 102 and the corresponding comfort material 202. To better illustrate an application of a preferred embodiment of the sternal brace 100, the torso 302 and the glenohumeral joint 304 are indicated.

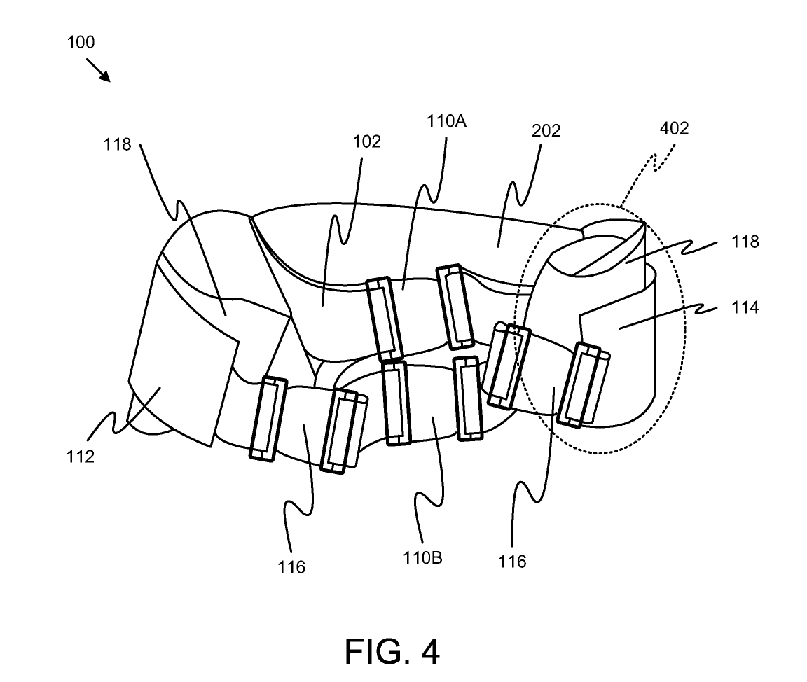

FIG. 4 is another view of one embodiment of the sternal brace 100 in accordance with the present invention. FIG. 4 illustrates the torso belt 102, the belt fastener 110, the humeral restraints 112, 114, restraint fasteners 116, the comfort material 118, and the comfort material 202. In one embodiment, the humeral restraints 112, 114 comprise the comfort material 118 with an adjustable cuff 402 circumscribing the corresponding upper arm. The depicted adjustable cuff 402 includes a portion of a hook and loop closure material such as Velcro®. Other closure material for the adjustable cuff 402 may also be used to secure the adjustable cuff 402 around an upper arm of a patient.

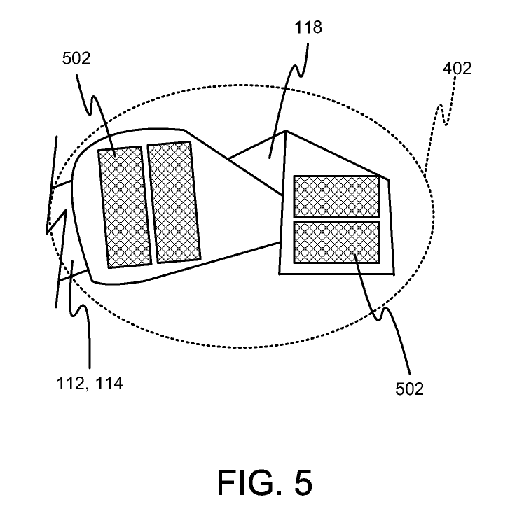

FIG. 5 is a more detailed depiction of the adjustable cuff 402 of the humeral restraint 112, 114 in accordance with the present invention. The adjustable cuff 402 comprises comfort material 118 and a hook and eye closure material 502, such as Velcro®. The opposing hook and eye closure material 502 is further illustrated.

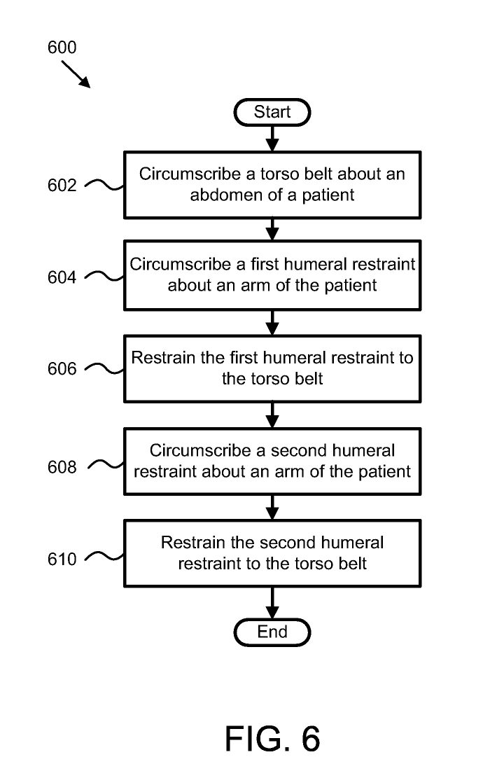

FIG. 6 is a schematic flow chart diagram illustrating an embodiment of a method 600 for assisting sternal rehabilitation. The method includes circumscribing 602 a torso belt 102 about the abdomen of a patient. The method 600 further includes circumscribing 604 a first humeral restraint 112 about an arm of the patient and restraining 606 the first humeral restraint 112 to the torso belt 102. Finally, the method 600 includes circumscribing 608 a second humeral restraint 114 about the other arm of the patient and restraining 610 the second humeral restraint 114 to the torso belt 102. One skilled in the art will recognize that circumscribing a belt or restraint necessarily requires securing the belt or restraint to itself.

One skilled in the art will further recognize that the order of steps is not critical so long as the patient's arms are held substantially in position by the side of the torso while the torso belt 102 is installed. For example, in certain embodiments, the first humeral restraint 112 is circumscribed 604 about the patient's arm before the torso belt 102 is circumscribed about the patient's abdomen. In other embodiments, the second humeral restraint 114 is circumscribed 608 about the patient's other arm before the first humeral restraint 112 is restrained 606 to the torso belt. These and other variations of the order of steps of the method are within the capabilities of one of skill in the art and within the scope of the present invention as defined by the claims below.

For patients recovering from a sternotomy, use of the present invention may provide important and beneficial sternal rehabilitation effects. By substantially removing the possibility of harmful accidental movement of the arms, confining the upper arms in an optimum healing orientation, and allowing simple movements of the lower arms, the patient may greatly increase the probability of successfully rehabilitating of the sternum. The Figures shown and described above are provided to illustrate one embodiment of the present invention. However, other embodiments are contemplated. For example, the torso belt 102 may comprise a single strap that circumscribes the upper arms and the torso 302 together with additional straps that pass between the arms and the abdomen 104. The sternal brace 100 may be further produced or implemented without the use of comfort materials. In any preferred embodiment, the sternal brace 100 may comprise any configuration of belts and/or straps that act to substantially confine the upper arm to a position slightly forward and directly to the side of the torso 302.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus to assist sternal rehabilitation, the apparatus comprising:
a torso belt circumscribing an abdomen of a patient;
a first humeral restraint coupled to the torso belt, the first humeral restraint circumscribing a first upper arm of the patient between a glenohumeral joint and an antecubital space of the first upper arm, the first humeral restraint substantially confining the first upper arm to a position lateral, slightly anterior, and to the side of a torso of the patient; and
a second humeral restraint coupled to the torso belt, the second humeral restraint circumscribing a second upper arm of the patient between a glenohumeral joint and an antecubital space of the second upper arm, the second humeral restraint substantially confining the second upper arm to a position lateral, slightly anterior, and to the side of the torso of the patient.

2. The apparatus of claim 1, wherein the torso belt comprises a substantially inelastic flexible material.

3. The apparatus of claim 2, wherein the torso belt further comprises a comfort material coupled to the substantially inelastic flexible material, the comfort material interposed between the substantially inelastic flexible material and the torso.

4. The apparatus of claim 1, wherein the torso belt circumscribes the abdomen below a pectoral line and above a pelvic line of the patient.

5. The apparatus of claim 1, wherein each humeral restraint positions a midpoint of each corresponding upper arm between a vertical mid-axillary line and a vertical line through a greater trochanter of a femur.

6. The apparatus of claim 1, wherein the torso belt comprises at least one belt fastener, the belt fastener securing the torso belt to the abdomen of the patient.

7. The apparatus of claim 1, wherein the length of the torso belt is adjustable.

8. The apparatus of claim 1, wherein each of the humeral restraints comprises a substantially inelastic flexible material.

9. The apparatus of claim 8, wherein each of the humeral restraints further comprises a comfort material coupled to the substantially inelastic flexible material, the comfort material interposed between the substantially inelastic flexible material and each upper arm.

10. The apparatus of claim 1, wherein each of the humeral restraints coupled to the torso belt comprises a restraint fastener, the restraint fastener further anchoring each of the humeral restraints to the torso belt thereby substantially securing the upper arm to the side of the torso.

11. The apparatus of claim 1, wherein the length of each of the humeral restraints is adjustable.

12. The apparatus of claim 1, wherein each of the humeral restraints comprise a cuff circumscribing the corresponding upper arm with a hook and loop closure material.

13. An apparatus to assist sternal rehabilitation, the apparatus comprising:
a torso belt circumscribing an abdomen of a patient, the torso belt comprising a substantially inelastic flexible material and the torso belt configured to circumscribe the abdomen between a pectoral line and a pelvic line of the patient; and
a first humeral restraint and a second humeral restraint coupled to the torso belt, wherein each humeral restraint
circumscribes an upper arm of the patient between a glenohumeral joint and an antecubital space of the upper arm;
substantially confines the upper arm to a position lateral, slightly anterior, and to the side of a torso of the patient; and
comprises a substantially inelastic flexible material.

14. A method for assisting sternal rehabilitation, the method comprising:
circumscribing a torso belt about an abdomen of a patient;
circumscribing a first humeral restraint about a first upper arm of the patient between a glenohumeral joint and an antecubital space of the first upper arm;
restraining the first humeral restraint to the torso belt, thereby substantially confining the first upper arm to a position lateral, slightly anterior, and to the side of a torso;
circumscribing a second humeral restraint about a second upper arm of the patient between a glenohumeral joint and an antecubital space of the second upper arm; and
restraining the second humeral restraint to the torso belt, thereby substantially confining the second upper arm to a position lateral, slightly anterior, and to the side of a torso.

15. The method of claim 14, wherein the torso belt comprises a substantially inelastic flexible material.

16. The method of claim 15, wherein a comfort material is interposed between the substantially inelastic flexible material and the torso.

17. The method of claim 14, wherein the torso belt circumscribes the abdomen below a pectoral line and above a pelvic line of the patient.

18. The method of claim 14, wherein a midpoint of each upper arm is restrained between a vertical mid-axillary line and a vertical line through a greater trochanter of a femur.

19. The method of claim 14, wherein each humeral restraint comprises a substantially inelastic flexible material.

20. The method of claim 19, wherein a comfort material is interposed between the substantially inelastic flexible material and each upper arm.
